(19) United States
(12) Patent Application Publication
(10) Pub. No.: US 2016/0346522 A1
(43) Pub. Date: Dec. 1, 2016

(54) BALLOON FOR MEDICAL DEVICE

(71) Applicant: Covidien LP, Mansfield, MA (US)

(72) Inventors: Kumin Yang, Plymouth, MN (US); Wang Yang, Brooklyn Park, MN (US); Bradley Steele, Plymouth, MN (US)

(21) Appl. No.: 14/727,267

(22) Filed: Jun. 1, 2015

(51) Int. Cl. A61M 25/10; B29C 49/04; B29C 49/00

(52) U.S. Cl. CPC A61M 25/1029; A61M 25/10; B29C 49/0005; B29C 49/04; A61M 2205/02; B29K 2077/00

(57) ABSTRACT

A balloon for a medical device is made from a biocompatible material including a base resin polymer and at least one additive totaling 1.0% or less by weight of the base resin polymer.

### FIELD OF THE DISCLOSURE

The present invention generally relates to a balloon for a medical device and a medical device including a balloon.

### BACKGROUND

Balloons mounted on the distal ends of catheters are widely used in medical treatment. The balloon may be used to widen a vessel into which the catheter is inserted, open a blocked vessel and/or deliver a medical device to a body location among other uses. In use, the uninflated balloon is delivered to a treatment location within a body lumen (e.g., a blood vessel) by tracking through an introducer sheath and exiting a distal end of the sheath to reach the treatment location. Once the uninflated balloon has reached the treatment location, fluid is delivered into the balloon, thereby expanding the outer circumference of the balloon (i.e., balloon is inflated). After treatment, the balloon is deflated and “pulled back” into the introducer sheath. The balloon catheter can then be withdrawn from the introducer sheath and the patient's body. It may be necessary or desired to re-introduce the balloon catheter into a body lumen, through the introducer sheath, to further treat the body lumen.

One known method of forming a medical balloon involves blow molding. In particular, the balloon is formed by radially expanding a segment of extruded polymer tubing, called a parison, in a mold. Typically, medical grade polymers used in forming balloons do not include additives. Balloons produced by radially expanding a parison may experience degradation of the polymer used to form the parison during extrusion or blow molding.

### SUMMARY

In one aspect, a balloon for a medical device includes a biocompatible material including a base resin polymer and at least one additive. The at least one additive totals 1.0% or less by weight of the base resin polymer.

### BRIEF DESCRIPTION OF THE DRAWINGS

FIG. 1 is a perspective of one embodiment of a medical balloon for a balloon catheter;

FIG. 2 is a fragmentary perspective of a balloon catheter including the balloon of FIG. 1;

FIG. 3 is a chart depicting data collected during straight line burst testing of test balloons and control balloons;

FIG. 4 is a chart depicting data collected during pull-back force testing, re-insertion force testing, and sheath compatibility testing of test balloons and control balloons;

FIG. 5 is a chart depicting data collected during fatigue testing of test balloons and control balloons;

FIG. 6 is a chart depicting data collected during tracking testing of test balloons and control balloons; and

FIG. 7 is a chart depicting data collected during outer diameter testing of test balloons and control balloons.

Corresponding reference characters indicate corresponding parts throughout the drawings.

### DETAILED DESCRIPTION OF EMBODIMENTS

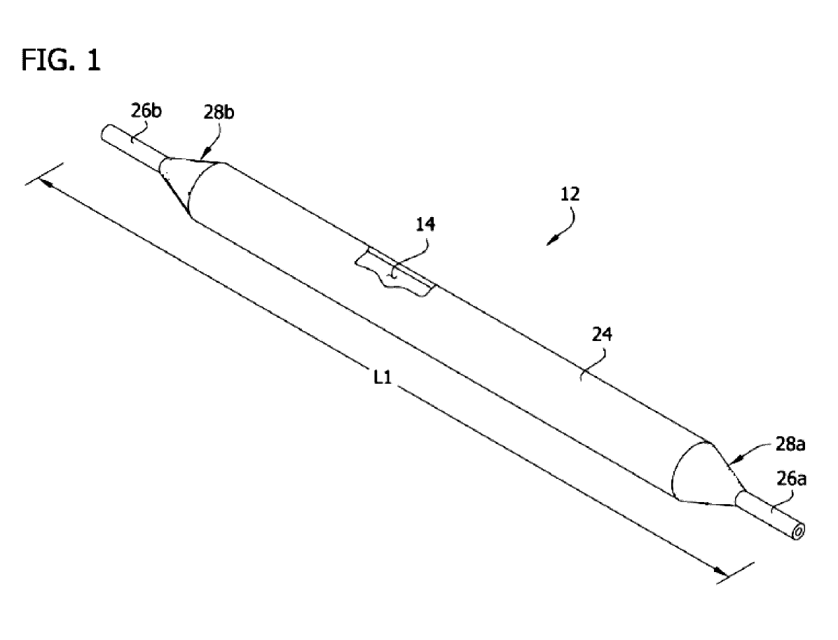

The present disclosure is directed to a transluminal balloon for a medical device. In one embodiment, the balloon is generally elongate and includes a generally tubular balloon body and cone segments at opposite longitudinal ends of the balloon body. The balloon defines an interior chamber for receiving fluid therein to expand an outer circumference of the balloon body. With respect to any or all of the below-described embodiments of the present disclosure, the transluminal balloon may be secured to a catheter (as shown in FIG. 1) or other medical device and configured for introduction along and inflation (or circumferential expansion) within a blood vessel for treating vascular stenosis. For example, the transluminal balloon may be configured for introduction along and inflation within one or more of peripheral arteries and veins, coronary arteries and veins, renal arteries and veins, cerebral arteries and veins, and carotid artery. In other examples, the transluminal balloon may be configured for introduction along and inflation within other body lumens for treating stenosis of those lumens.

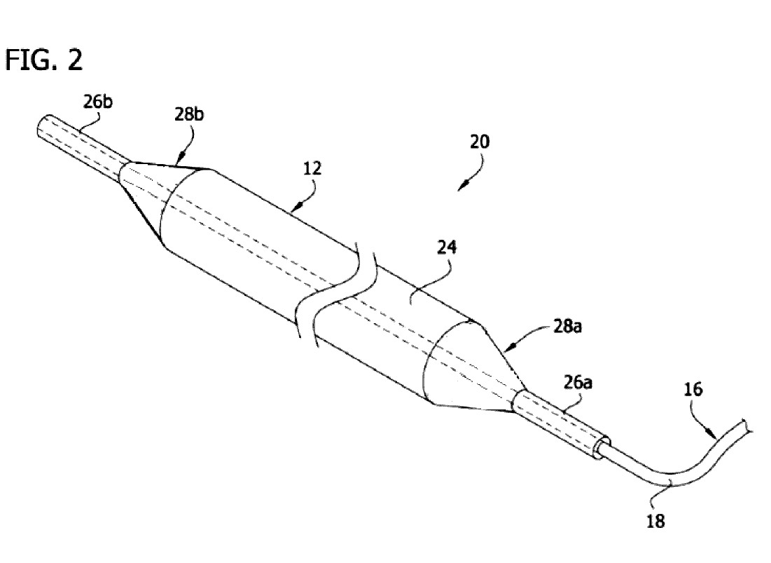

Referring to FIGS. 1 and 2, one embodiment of a medical balloon for a medical device is generally indicated at reference numeral 12 in FIG. 1. The balloon defines an interior chamber 14 for receiving fluid therein to expand an outer circumference (i.e., an outer periphery) of the balloon. The balloon 12 is shown in its expanded or inflated configuration throughout the drawings, with the understanding that in its uninflated and deflated configurations, the balloon is capable of folding lengthwise such that the outer circumference of the balloon in its uninflated and deflated configurations is substantially less than the outer circumference of the balloon in its expanded configuration. With respect to any or all of the below described embodiments of the present disclosure, the medical balloon 12 may be secured to a catheter, generally indicated at 16 in FIG. 2, such that a catheter body 18 of the catheter extends axially through the interior chamber 14 of the balloon, as is generally known in the art, to form a balloon catheter, generally indicated at 20. The balloon 12 and catheter body 18 have suitable shapes and dimensions for introduction into a desired body lumen for treatment therein. Typically, the balloon 12, in its uninflated initial configuration, is introduced into the body lumen using an introducer sheath (not shown). The uninflated balloon 12 is delivered to a treatment location within a body lumen (e.g., a blood vessel) by tracking through the introducer sheath and ultimately exiting a distal end of the sheath to reach the treatment location. Once the uninflated balloon 12 has reached the treatment location, fluid (e.g., saline) is delivered into the balloon to inflate or expand the balloon to a desired internal pressure, thereby expanding the outer circumference of the balloon. After treatment, the balloon 12 is deflated and “pulled back” into the introducer sheath. The balloon catheter 20 can then be withdrawn from the introducer sheath and the patient's body. It may be necessary or desired to re-introduce the balloon catheter 20 into a body lumen, through the introducer sheath, to further treat the body lumen.

The illustrated balloon catheter 20 may be configured for introduction along and inflation (i.e., circumferential or peripheral expansion) within a blood vessel for treating vascular stenosis. As an example, the medical balloon 12 of the illustrated balloon catheter 20 may be configured for introduction along and inflation within one or more of peripheral arteries and veins, coronary arteries and veins, renal arteries and veins, cerebral arteries and veins, and carotid artery. In other examples, the medical balloon 12 may be configured for introduction along and inflation within other body lumens for treating stenosis of those lumens. The balloon 12 may be configured for treating other body lumens and/or for other treatments of those lumens.

Referring to FIG. 1, the medical balloon 12 has a length L1 and comprises a balloon body section 24; opposite proximal and distal waist sections 26a, 26b, respectively, at opposite longitudinal ends of the balloon; and opposite proximal and distal cone sections, generally indicated at 28a, 28b, respectively, at corresponding proximal and distal ends of the body section intermediate the body section and the corresponding proximal and distal waist sections. It is understood that the balloon 12 may have other sections, structures, and/or components without departing from the scope of the present invention.

In one embodiment, the balloon 12 is formed from a polymer material that includes additives. For example, the balloon 12 may be formed from a suitable polymer base resin having less than or equal to 1.0% by weight of additives. In one embodiment, the balloon 12 is formed from a polymer base resin having additives in an amount in the range of about 0.005% by weight to 1.0% by weight. In one embodiment, the balloon 12 is formed from a polymer base resin having additives in an amount in the range of about 0.01% by weight to 1.0% by weight. The additives may include at least one of a thermal stabilizer, a UV/light stabilizer, a processing aid, and a plasticizer. In one embodiment, the balloon 12 is formed from a material free from plasticizer. In one embodiment, the balloon 12 is formed with a plasticizer as an additive, but the base resin is not plasticized. The additives can be selected and configured to target a single polymer or multiple polymers in the base resin. The balloon 12 can further include organic fillers (e.g., carbon nanotubes, graphenes, carbon fibers, etc.) and/or inorganic fillers (e.g., silica, etc.). For example, suitable polymer base resins for the balloon include thermoplastic polymers, thermoplastic elastomer polymers, polyesters such as PET, PEN and PBT; polyurethane block copolymers such as ISOPLAST 301, PELLETHANE 2363-75D; polyamide block copolymers such as PEBAX 6333, PEBAX 7033 and PEBAX 7233; polyamides such as nylon 12, nylon 11, and nylon 10; polymer blend materials such as single or multiphase blends of liquid crystal polymers in another polymer; and polyester elastomer balloons such as ARNITEL EM 740 and HYTREL 8238. Suitable thermal stabilizers for the balloon include, but are not limited to, copper compounds (e.g., copper iodide), potassium iodide, N, N′-hexamethylenebis-3-(3,5-ditertiarybutyl1-4-hydroxyphenyl)propionamide, alkylated diphenyl amines, and cyclic neopentanetetraylbis(2,6-di-t-butyl-4-methylphenyl) phosphite. Suitable UV/light stabilizers for the balloon include, but are not limited to, ultraviolet light absorbers (e.g., carbon black, rutile titanium oxide, hydroxybenzophenone, hydroxyphenylbenzotriazole, or oxanilides), quenchers, 2,2-methylenebis[4-(1,1,3,3,-tetramethyl)-6-(2H-benzotraol-2-yl) phenol], 2-(2′-hydroxy-3′,5′-dibenzylphenyl) benzotriazole, and Hindered Amine Light Stabilizers (HALS) (e.g., chemical compounds including 2,2,6,6-tetramethylpiperidine ring structure). Suitable processing aids for the balloon include, but are not limited to, fluorine-containing compounds (e.g., flouropolymers), N,N′-ethylenebisstearamide, N-stearylerucamide, stearyl alcohol, ethylenebisstearamide, polytetrafluoroethylene (PTFE), and silicone fluids. Suitable plasticizers for use as an additive for the balloon include, but are not limited to, N-butylbenzene sulfonamide and 2-ethylhexyl 4-hydroxybenzoate. Other additives are within the scope of the present invention.

In one embodiment, the balloon 12 is formed from a material that is biocompatible, such as a material that meets the requirements of USP Plastic Class VI. For example, the balloon 12 may be formed from a plastic that meets the requirements of USP Plastic Class VI, includes less than or equal to 1.0% by weight of additives, and is free from plasticizer. In one example, the balloon 12 may be formed from a plastic that meets the requirements of USP Plastic Class VI and includes less than or equal to 1.0% by weight of additives. The additives can include a plasticizer, but in an amount less than or equal to 1.0% by weight (i.e., the balloon includes a plasticizer but is not plasticized). Other materials do not depart from the scope of the present invention as defined by the claims. In one example, the balloon 12 may be free from a lubricious coating (hydrophobic or hydrophilic), although in other examples the balloon may include such a lubricious coating.

In one embodiment, the balloon 12 is formed from a nylon 12 polymer base resin that includes less than or equal to 1.0% by weight of additives. The balloon 12 can be free from plasticizer. The additives include a thermal stabilizer and a processing aid. The thermal stabilizer acts to capture and neutralize any free radicals generated due to high temperature (e.g., during extrusion) and/or high shear/elongational stress (e.g., during extrusion and during balloon forming). Polymer degradation before and after processing is minimized by reducing the free radicals. The processing aid or lubricant creates slippage between highly entangled polymer chains during melt (extrusion), solid, and pseudo solid/melt (balloon forming) stages. Both the thermal stabilizer and the processing aid reduce the friction between the polymer chains (i.e., the bulk polymer modulus is reduced). It is believed this softening favorably leads to the ability to fold the balloon 12 to a smaller profile for lower pull-back and re-insertion forces. This favorable folding is achieved without any significant negative impact on the mechanical properties of the balloon.

A suitable, non-limiting example of a nylon 12 base resin that is free from plasticizer and has less than or equal to 1.0% by weight of additives including a thermal stabilizer and a processing aid includes VESTAMID® CareML24 (previously VESTAMID® L2140), Acommercially available from Evonik Industries AG of Germany. The properties for VESTAMID® CareML24, commercially available from Evonik Industries AG of Germany, are provided in Table 1 (below).

TABLE 1

| | Typical | | |
|---|---|---|---|
| Property | Value | Unit | Test Method |
| Density 23° C. | 1.01 | g/cm³ | ISO 1183 |
| Water Absorption | 1.6 | % | |
| 23° C., saturation | | | |
| Water Absorption | 0.7 | % | ISO 62 |
| 23° C., 50% relative humidity | | | |
| Melting range DSC, 2nd heating | 178 | ° C. | ISO 11357 |
| Vicat softening temperature | | | |
| Method A 10 N | 170 | | |
| Method B 50 N | 140 | ° C. | ISO 306 |
| Linear thermal expansion | 1.4 | 10⁻⁴K⁻¹ | ISO 11359 |
| 23-55° C. | | | |
| Flammability acc. UL94 | | | |
| 3.2 mm | HB | | |
| 1.6 mm | HB | | IEC 60695 |
| Mold shrinkage (on 2 mm sheets, | | | |
| with film gate at rim, mold temp. | | | |
| 80° C.) | | | |
| in flow direction | 0.65 | % | |
| in transverse direction | 1.25 | % | ISO 294-4 |
| Temperature of deflection under load | | | |
| Method A 1.8 MPa | 50 | ° C. | |
| Method B 0.45 MPa | 110 | ° C. | ISO 75-1/-2 |
| Tensile Test | | | |
| Stress at yield | 47 | MPa | |
| Strain at yield | 5 | % | ISO 527-1 |
| Nominal strain at break | >50 | % | ISO 527-2 |
| Tensile Modulus | 1400 | MPa | ISO 527-1/-2 |
| Charpy Impact | | | |
| Unnotched 23° C. | No Break | kJ/M² | ISO 179/1eU |
| Unnotched −30° C. | No Break | kJ/M² | |
| Notched 23° C. | 16 C. | kJ/M² | ISO 179/1eA |
| Notched −30° C. | 9 C. | kJ/M² | |

Testing was conducted to compare the performance of balloons formed from a polymer without additives to the performance of balloons formed from a polymer with additives. Test balloons were formed from VESTAMID® CareML24 (previously VESTAMID® L2140), a nylon 12 base polymer with a thermal stabilizer and a processing aid. Control balloons were formed from GRILAMID® L25, a nylon 12 polymer without additives, commercially available from EMS-GRIVORY of Switzerland. The inflated diameters of the body sections of the test and control balloons measured 8 mm and the lengths of the test and control balloons measured 100 mm (i.e., 8×100 mm balloons). The two groups of balloon were produced with similar balloon double wall thickness, and full catheters were manufactured and EtO sterilized.

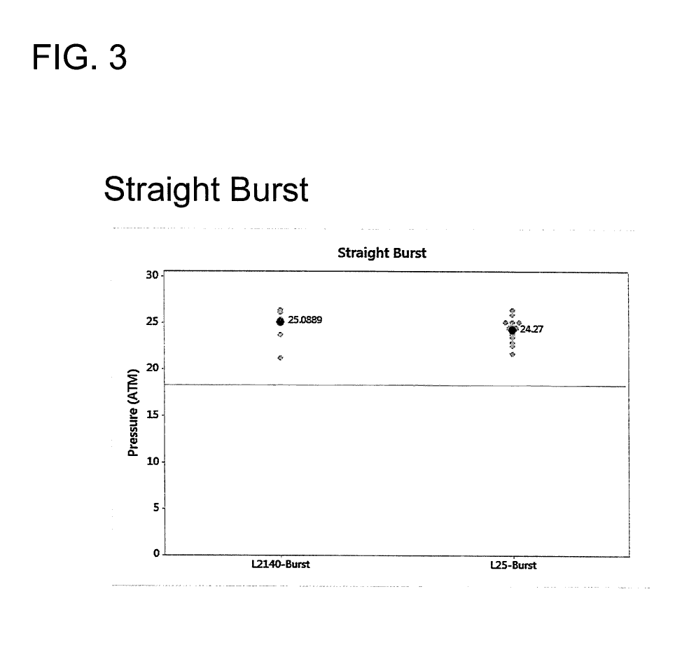

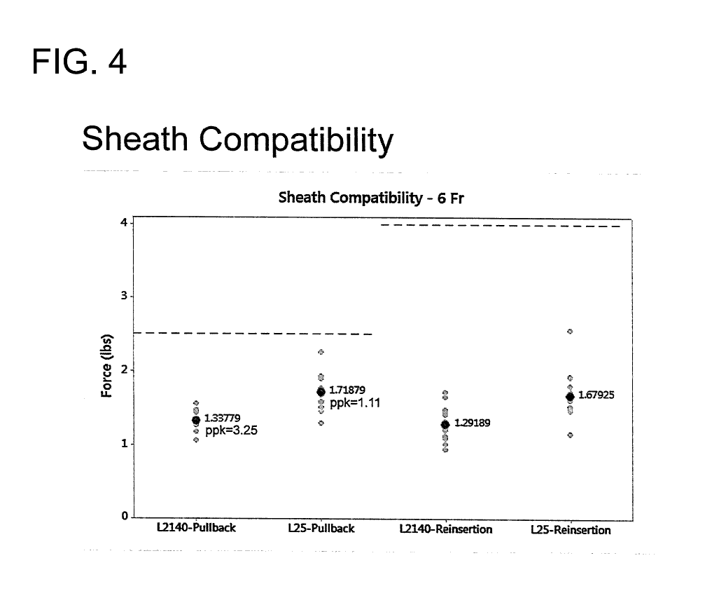

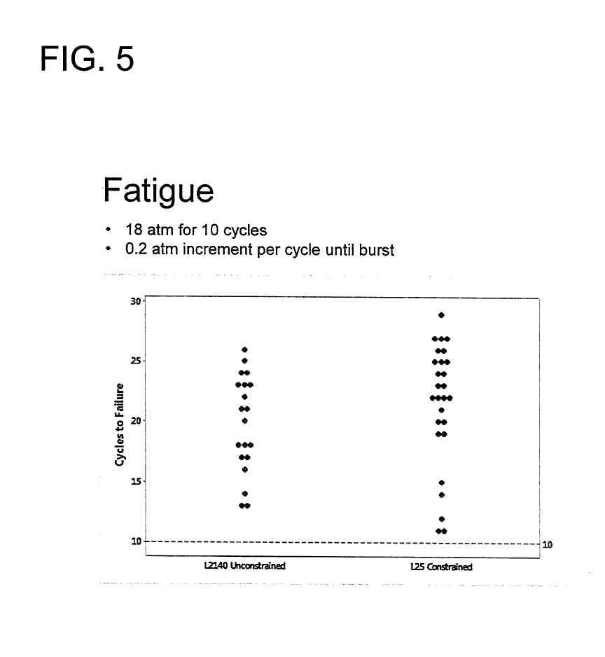

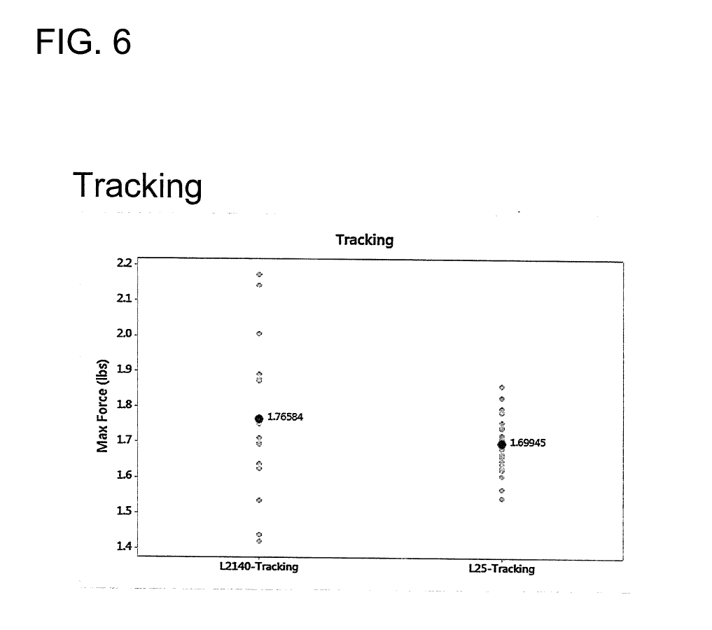

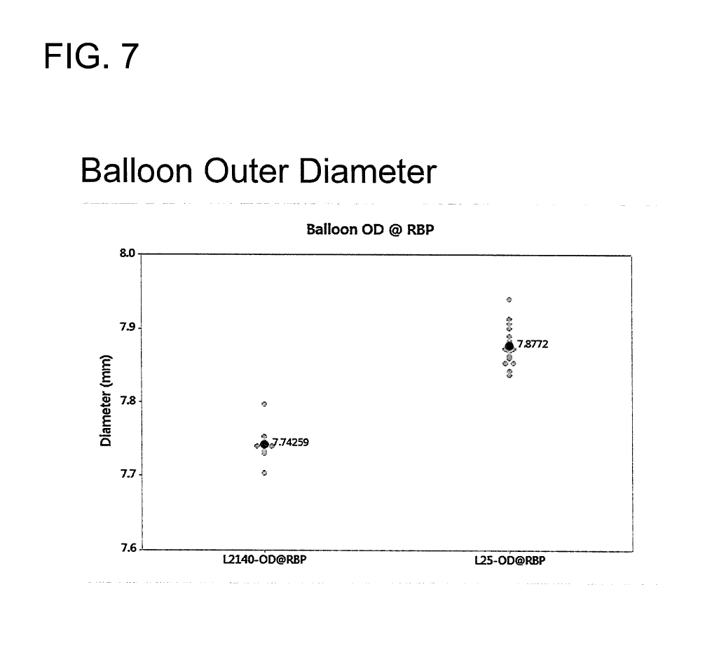

Each of the balloons from the test and control groups was tested for the straight line burst pressure. The overall results of the straight line burst pressure test are shown in the chart provided in FIG. 3. Each of the balloons from the test and control groups was tested for the pull-back force required to pull the balloon, in its deflated configuration, back into a distal end of an introducer sheath, and for sheath compatibility during pull-back. The overall results of the pull-back force test and sheath compatibility test are shown in the chart provided in FIG. 4. Each of the balloons from the test and control groups was also tested for the re-insertion force required to re-insert the deflated balloon into the introducer sheath, and for sheath compatibility during re-insertion. The overall results of the re-insertion force test and sheath compatibility are also shown in the chart provided in FIG. 4. Each of the balloons from the test and control groups was also tested for fatigue by inflating to 18 atm for 10 cycles and then increasing in 0.2 atm increments per cycle until burst. The overall results of the fatigue test are shown in the chart provided in FIG. 5. Each of the balloons from the test and control groups was also tested for trackability by recording the maximum force required to traverse a simulated contralateral peripheral intervention approach. The overall results of the tracking test are shown in the chart provided in FIG. 6. The balloon outer diameters of each of the test and control group balloons were measured at 18 atm. The overall results of the balloon outer diameter test are shown in the chart provided in FIG. 7.

The average results obtained in the testing of the test and control group balloons are provided in Table 2 (below).

TABLE 2

| | Test Group | Control Group |
|---|---|---|
| | (VESTAMID ® | (GRILAMID ® |
| | CareML24/L2140) | L25) |
| Mold Diameter (inch) | 0.284 | 0.284 |
| DWT (avg, inch) | 0.00297 | 0.00285 |
| Balloon Outer Diameter at | 7.74 | 7.88 |
| 18 atm (mm) | | |
| Balloon Length at 18 atm (mm) | 100 | 99.7 |
| Pull-back Force (lbs) | 1.34 | 1.72 |
| Re-insertion Force (lbs) | 1.29 | 1.68 |
| Straight Burst (atm) | 25.1 | 24.3 |

As can be seen from Table 2 and FIGS. 3-7, the pull-back force and the re-insertion force for the test balloon was significantly less than the control balloon, showing that the additives in the test balloon material contribute to the reduced pull-back and re-insertion force. Surprisingly, no significant differences in the straight line burst pressures were observed, and the fatigue results were comparable.

In one embodiment, the balloon 12 is formed by blow molding a parison. The parison comprises the material as disclosed above. The parison may be formed by extrusion or other methods. The parison is inserted into a mold and blow molded to form the balloon 12. The process of blow molding may be a conventional process well known in the art. The balloon 12 may be formed in other ways.

Having described the invention in detail, it will be apparent that modifications and variations are possible without departing from the scope of the invention defined in the appended claims.

When introducing elements of the present invention or the preferred embodiment(s) thereof, the articles “a”, “an”, “the” and “said” are intended to mean that there are one or more of the elements. The terms “comprising,” “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A balloon for a medical device, the balloon comprising a biocompatible material including a base resin polymer and at least one additive, the at least one additive totaling 1.0% or less by weight of the base resin polymer.

2. The balloon set forth in claim 1, wherein the at least one additive is selected from a group consisting of a thermal stabilizer, a UV stabilizer, a processing aid, and a plasticizer.

3. The balloon set forth in claim 2, wherein the base resin polymer comprises nylon 12.

4. The balloon set forth in claim 1, wherein the at least one additive comprises a thermal stabilizer and a processing aid.

5. The balloon set forth in claim 1, wherein the biocompatible material is free from plasticizers.

6. The balloon set forth in claim 1, wherein the at least one additive reduces the bulk modulus of the base resin polymer.

7. The balloon set forth in claim 6, wherein the at least one additive is selected from a group consisting of a thermal stabilizer, a UV stabilizer, and a processing aid.

8. The balloon set forth in claim 6, wherein the base resin polymer comprises nylon 12.

9. The balloon set forth in claim 6, wherein the at least one additive comprises a thermal stabilizer and a processing aid.

10. The balloon set forth in claim 6, wherein the biocompatible material is free from plasticizers.

11. The balloon set forth in claim 1, wherein the at least one additive comprises a thermal stabilizer and a processing aid, and the biocompatible material is free from plasticizers.

12. The balloon set forth in claim 11, wherein the base resin polymer comprises nylon 12.

13. The balloon as set forth in claim 1, wherein the at least one additive is targeted to one or more polymer in the base resin polymer.

14. The balloon as set forth in claim 1, wherein the balloon is sized and shape for introduction into a blood vessel.

15. The balloon as set forth in claim 14, in combination with a catheter secured to the balloon.

16. A method of forming a balloon for a medical device, comprising:
providing a parison, the parison comprising a biocompatible material including a base resin polymer and at least one additive, the at least one additive totaling 1.0% or less by weight of the base resin polymer,
blow molding the parison to form the balloon.

17. The method of forming a balloon as set forth in claim 16, wherein said providing a parison comprises extruding the parison.

18. The method of forming a balloon as set forth in claim 16, wherein the at least one additive is selected from a group consisting of a thermal stabilizer, a UV stabilizer, a processing aid, and a plasticizer.

19. The method of forming a balloon as set forth in claim 18, wherein the base resin polymer comprises nylon 12.

20. The method of forming a balloon as set forth in claim 16, wherein the at least one additive is targeted to one or more polymer in the base resin polymer.
